(19) United States
(12) Patent Application Publication
(10) Pub. No.: US 2025/0191742 A1
(43) Pub. Date: Jun. 12, 2025

(54) APPARATUSES, SYSTEMS, AND METHODS FOR DETERMINING HEALTHCARE VITALITY

(71) Applicant: Healthcare TTU, Inc., Littleton, CO (US)

(72) Inventors: Travis W. Gentry, Highlands Ranch, CO (US); Timothy R. Estes, Castle Rock, CO (US); Jae R. Waldron, Murfreesboro, TN (US); Dale Ray Alexander, Montgomery, AL (US); Michael J. Cunningham, Littleton, CO (US)

(21) Appl. No.: 19/057,859

(22) Filed: Feb. 19, 2025

Related U.S. Application Data
(63) Continuation of application No. 17/541,004, filed on Dec. 2, 2021, now abandoned.
Application No. 17/541,004 is a continuation of application No. PCT/US2020/037590, filed on Jun. 12, 2020.
(60) Provisional application No. 62/860,317, filed on Jun. 12, 2019.

(51) Int. Cl. G16H 40/20; G06Q 10/0635; G06Q 10/0639; G06Q 40/08

(52) U.S. Cl. CPC G16H 40/20; G06Q 10/0635; G06Q 10/06393; G06Q 40/08

(57) ABSTRACT

Systems, apparatuses, and methods are provided for determining healthcare vitality. A healthcare provider device may transmit a set of provider data to a server. The server may include a de-identification section to de-identify at least a portion of the received set of provider data, a matching section to match the at least a portion of the received set of provider data having been de-identified to create a set of matched de-identified data, a processing section to process the matched de-identified data to extract at least one set of coded information according to a predetermined data format of the set of provider data, a categorization section to categorize the at least one set of coded information to one or more categories associated with the healthcare provider, and a vitality scoring section to determine at least one vitality composite score.

### CROSS-REFERENCES TO RELATED APPLICATIONS

This application is a continuation of U.S. patent application Ser. No. 17/541,004, filed Dec. 2, 2021, entitled “Apparatuses, Systems, and Methods for Determining Healthcare Vitality,” which claims benefit of Patent Cooperation Treaty (PCT) Application No. PCT/US2020/037590, filed Jun. 12, 2020, entitled “Apparatuses, Systems, and Methods for Determining Healthcare Vitality,” which claims benefit of U.S. Provisional Patent Application No. 62/860,317, filed Jun. 12, 2019, entitled “Apparatuses, Systems, and Methods for Determining Healthcare Vitality,” each of which is hereby incorporated by reference in its entirety.

### TECHNICAL FIELD

The present disclosure relates generally to apparatuses, systems, and methods for determining healthcare vitality.

### FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND

The healthcare industry experiences problems relating to the visibility of pricing and performance data. Think of a world where commercial retailers like Wal-Mart, Target, or BestBuy have no idea if their wholesale cost of a Samsung TV is competitive because the pricing Samsung offers each retailer is unique and confidential. Think of a world where the consumer has no idea how much a television will cost until they receive a bill weeks or months after the sale. This is currently the world of healthcare! Now think of a world where people invest money into the stock market and attempt to create a diversified portfolio of different stocks, bonds, and mutual funds. Think of a world where there is no competitive information as to the historical or current performance of those investments and no means to evaluate or measure risk, return, or performance. This is the world of healthcare.

Healthcare is an industry that is deathly afraid of true transparency; however, transparency is healthcare's single greatest hope for real transformation. The industry operates in a world where providers engage in meaningless “charge” conversations. Reimbursement is hidden under a veil of secrecy by both insurance companies and providers alike. Health insurance companies have all the information while providers are disadvantaged and negotiate without any idea if their reimbursement is competitive. Providers lack access to real-time, unbiased, industry benchmarks and peer comparisons. Providers operate under a false sense of security and really don't know how well their operations are performing compared to their peers.

### SUMMARY

Embodiments of the present invention provide apparatuses, systems, and methods for determining healthcare operational and process vitality.

Hospitals, physicians, patients, and financial institutions desperately need a marketplace where meaningful information is readily available. Hospitals and physicians need to know how well their performance compares to the industry and peers. They need to know if their reimbursement is fair and equitable. Financial institutions need to understand the financial health, strength, and data of the healthcare industry they serve. None of these are possible unless healthcare as an industry is willing to share their information in an anonymous, safe, and secure fashion. Healthcare data needs to be aggregated and benchmarked to empower providers and financial institutions to compare and contrast performance.

How good are hospital managed care contracts? How do hospitals know how good they are? How does their reimbursement compare to the industry, compare against their competition? Is Aetna paying their competitor better for orthopedic care? Insurance companies dictate their manage care contract reimbursement rates are intentionally confidential and are contractually restricted from industry transparency. Providing national/peer blinded and aggregated percentile reimbursement statistics would be the only way to truly determine for a provider where the reimbursement they receive for the procedures performed compares.

Apparatuses, systems, and methods consistent with the present disclosure address these and other needs in the art in a novel and nonobvious manner.

According to aspects of the present disclosure, provided is a system for determining healthcare vitality. The system includes a network, a healthcare provider device configured to transmit a set of provider data via the network, and a server having a storage. The server receives the set of provider data via the network. The server includes a de-identification section which performs a de-identification operation on at least a portion of the received set of provider data, a matching section which performs a matching operation on the at least a portion of the received set of provider data having been de-identified by the de-identification section to create a set of matched de-identified data, a processing section which processes the matched de-identified data to extract at least one set of coded information according to a predetermined data format of the set of provider data, a categorization section which examines at least a portion of the at least one set of coded information and to categorize the at least one set of coded information to one or more categories associated with the healthcare provider, a vitality scoring section which determines at least one vitality composite score based at least in part upon the categorized at least one set of coded information, and a communications unit which selectively transmits at least a set of information relating to the at least one vitality composite score. The storage of the server may store a representation of at least a portion of the set of provider data.

The categorization section may associate one or more processed categories with a plurality of healthcare providers or payers. The one or more processed categories may relate to a vitality score for a healthcare provider associated with the at least one vitality composite score. The system may include a user device having a communications unit and a display unit. The user device may be coupleable to the network. The user device may communicate with the server via the network to obtain information relating to the vitality score. The user device may provide a representation of the information relating to the vitality score to a user of the user device via the display unit.

The server may include a normalization section which performs a normalization operation on the at least one set of coded information in association with the vitality score to form a set of normalized provider data. The user device may display the normalized provider data via the display unit. The user device may display the normalized provider data for a plurality of healthcare providers via the display unit. The categorization section may categorize the at least a portion of the matched de-identified data to at least one category which is non-coded with respect to a data format of the set of provider data.

The server may include a normalization section which performs a normalization operation on the at least one set of coded information. The normalization operation may be a localized cost adjustment.

According to additional aspects of the present disclosure, provided is a method for determining healthcare vitality. The method includes receiving a set of provider data from a healthcare provider, performing a de-identification operation on at least a portion of the received set of provider data, creating a set of matched de-identified data by performing a matching operation on the at least a portion of the received set of provider data, processing the matched de-identified data to extract at least one set of coded information according to a predetermined data format of the set of provider data, examining at least a portion of the matched de-identified data and categorizing the at least a portion of the coded information to one or more categories associated with the healthcare provider, determining at least one vitality composite score based at least in part upon the categorized at least one set of coded information, and selectively transmitting or storing at least a set of information relating to the at least one vitality composite score.

The method may include associating one or more processed categories with a plurality of healthcare providers or payers, wherein the one or more processed categories relate to a vitality score for the plurality of healthcare providers.

The method may further include obtaining information relating to the vitality score at a user device, and visually displaying the information relating to the vitality score to a user of the user device.

The method may further include normalizing the at least one set of coded information in association with the vitality score to form a set of normalized provider data.

The method may further include transmitting at least a portion of the set of normalized provider data to a user device, and visually displaying the at least a set of normalized provider data by the user device.

According to yet other aspects of the present disclosure provided is an apparatus for determining healthcare vitality. The apparatus includes a storage, a communications section coupleable to a network, the communications section configured to receive a set of provider data, a de-identification section configured to perform a de-identification operation on at least a portion of the received set of provider data, a matching section configured to perform a matching operation on the at least a portion of the received set of provider data having been de-identified by the de-identification section to create a set of matched de-identified data, a processing section configured to process the matched de-identified data to extract at least one set of coded information according to a predetermined data format of the set of provider data, a categorization section configured to examine at least a portion of the at least one set of coded information and to categorize the at least one set of coded information to one or more categories associated with the healthcare provider, and a vitality scoring section configured to determine at least one vitality composite score based at least in part upon the categorized at least one set of coded information. The storage may store a representation of at least a portion of the set of provider data.

The categorization section may associate one or more processed categories with a plurality of healthcare providers or payers. The one or more processed categories may relate to a vitality score for a healthcare provider associated with the at least one vitality composite score. The apparatus may include a normalization section, the normalization section configured to perform a normalization operation on the at least one set of coded information in association with the vitality score to form a set of normalized provider data.

Numerous other objects, features, and advantages of the present invention will be readily apparent to those skilled in the art upon a reading of the following disclosure when taken in conjunction with the accompanying drawings.

### BRIEF DESCRIPTION OF THE DRAWINGS

FIG. 1 illustrates an exemplary embodiment of computer system-level implementation consistent with the present disclosure.

FIG. 2 illustrates a functional network diagram of an exemplary embodiment of a system according to aspects of the present disclosure.

FIG. 3 illustrates an exemplary embodiment of report data tables according to aspects of the present disclosure.

FIG. 4 illustrates an exemplary embodiment of a table structure including pre-calculated scores according to aspects of the present disclosure.

FIG. 5 illustrates an exemplary embodiment of a process for processing provider data according to aspects of the present disclosure.

FIG. 6 illustrates an exemplary embodiment of a partial block diagram of a server according to aspects of the present disclosure.

FIGS. 7A-7H together illustrate an exemplary embodiment of a Vitality Score composite along with velocity index, value index, variety index, and volatility index information and corresponding raw data according to aspects of the present disclosure.

FIG. 7A illustrates an exemplary embodiment of a Vitality Composite and Velocity Index information table according to aspects of the present disclosure.

FIG. 7B illustrates an exemplary embodiment of a Velocity Index Data table according to aspects of the present disclosure.

FIG. 7C illustrates an exemplary embodiment of a Value Index table according to aspects of the present disclosure.

FIG. 7D illustrates an exemplary embodiment of a Value Index Data table according to aspects of the present disclosure.

FIG. 7E illustrates an exemplary embodiment of a Variety Index table according to aspects of the present disclosure.

FIG. 7F illustrates an exemplary embodiment of a Variety Index Data table according to aspects of the present disclosure.

FIG. 7G illustrates an exemplary embodiment of a Volatility Index table according to aspects of the present disclosure.

FIG. 7H illustrates an exemplary embodiment of a Volatility Index Data table according to aspects of the present disclosure.

FIGS. 8A-8F together illustrate an exemplary embodiment of a vector scoring metric scenario 800 including an ATEX Vector Score, a volume composite, a velocity composite, a value composite, a variety composite, and a volatility composite, along with sub-components thereof for each composite value according to aspects of the present disclosure and consistent with the present disclosure.

FIG. 8A illustrates an exemplary embodiment of a Vector Score and Volume Composite table according to aspects of the present disclosure.

FIG. 8B illustrates an exemplary embodiment of a Velocity Composite table according to aspects of the present disclosure.

FIG. 8C illustrates an exemplary embodiment of a Value Composite table according to aspects of the present disclosure.

FIG. 8D illustrates an exemplary embodiment of a Variety Composite table according to aspects of the present disclosure.

FIG. 8E illustrates an exemplary embodiment of a Volatility Composite table according to aspects of the present disclosure.

FIG. 8F illustrates an exemplary embodiment of data and graphical information useable according to aspects of the present disclosure.

FIGS. 9A-9D together illustrate an exemplary embodiment of Volatility Index calculation and presentation capabilities 900 to a user according to aspects of the present disclosure.

FIG. 9A illustrates an exemplary embodiment of a chart relating to hospital metrics and benchmark averages provided according to aspects of the present disclosure.

FIG. 9B illustrates an exemplary embodiment of a chart relating to hospital metrics and benchmark averages provided according to aspects of the present disclosure.

FIG. 9C illustrates an exemplary embodiment of Volatility Index ideas and corresponding metrics according to aspects of the present disclosure.

FIG. 9D illustrates an exemplary embodiment of weights, metrics, benchmark averages and a fluctuations index according to aspects of the present disclosure.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

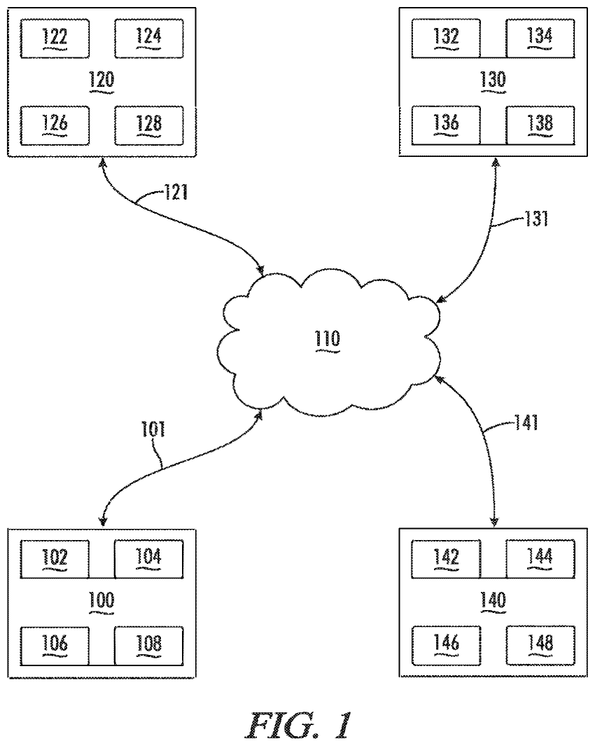

Referring generally to FIGS. 1-9, various exemplary apparatuses, systems, and methods are provided according to aspects of the present disclosure.

Implementations consistent with the present disclosure may provide an ATEX Vitality Score™ as an online comparison website built for the healthcare and financial industries. Like a consumer credit score, an ATEX Vitality Score™ may measure one or more of Velocity, Value, Variety, and/or Volatility indices of healthcare operational performance and payer reimbursement. Implementations consistent with the present disclosure may enable a transparent marketplace to compare and benchmark providers with actionable analytics and meaningful metrics.

In various exemplary embodiments, the ATEX Vitality Score™ may be configured in such a way as to aggregate individual hospital information in a centralized website which allows a hospital to compare their contracted reimbursement rates against other hospitals and health systems across the industry. Hospitals now have visibility to inpatient and outpatient reimbursement by facility, by payer, and/or by specialty. They may be able to benchmark the reimbursement by payer and determine if their contracted rates by specialty are comparable, better, or worse than their competitors.

Individual hospitals and health systems data, including 837 file data, 835 file data, and BTRS data may address claims, remits, and bank records. This large volume of data may accrue for each hospital. Implementations consistent with the present disclosure may provide a national comparison portal which is capable of providing benchmarking of such data, of aggregating and de-identifying the data, and determining a Vitality Score associated with the data. One or more user interfaces may be provided to present processed data and comparisons and to function as a data custodian.

Healthcare professionals, bankers, investors, lenders, consultants, etc. may be able to subscribe to the ATEX Vitality Score™ where healthcare provider information may be searchable and comparable. Healthcare professionals belonging to a hospital or health system may be able to compare their specific hospital against deidentified data of other anonymous hospitals. They may be able to compare their hospitals to others by using generic demographic and geographic data such as bed size, net patient revenue, hospital type, payer mix, etc.

The ATEX Vitality Score™ may be configured to provide a normalized standard to assess the cash performance of a specific healthcare entity across payers, and business lines with national averages and blinded peer performance. This assessment may be across one or more of a plurality of measures.

Benefits—The ATEX Vitality Score™ has several clear advantages to existing analytic platforms.
(1) Impartial Measures—many of the existing analytic platforms available are associated with a vendor providing other services, often the analytics are related to proving the usefulness of this vendors services. The score may be from an independent and impartial party not concerned in how the scores might reflect on a vendor's performance.
(2) Common Data format-many of today's analytic platforms are dependent on usage of a specific vendor and their specific data feeds and formats. The ATEX Vitality Score™ might use only standard industry data sources that do not involve sensitive HIPAA or EHR data.
(3) Peer and National Comparisons-some of today's top analytics platforms do offer a scoring system with a national average based on their install-base, but only ATEX Vitality Score™ is an industry solution designed to allow subscribers to submit their own data and to compare against customizable peer groups of similar size, region, or specialty.
(4) Comparing Specific Measures-other top analytics platforms do offer comparison to total scoring, but only ATEX Vitality Reimbursement Comparative Analytics allows you to compare specific metrics from DNFB to Denial percentages with peer groups.
(5) Clear and intuitive comparisons-many existing analytic platforms compare specific numbers on a special scale of their own creation. Unlike these existing analytic platforms, implementations consistent with the present disclosure may include, for example, ATEX Vitality Reimbursement Comparative Analytics having clear intuitive percentile rankings for metrics.

### Payer Reimbursement and Denial Comparison

Payer Comparison—A measure of top payers in Commercial and Government categories may be provided using metrics and/or a percentile rank comparison, for both inpatient and outpatient procedures anonymously presented by hospitals as a percentile of each other. Comparisons may be performed at a general hospital level, a national level, a peer group level, and may extend across a plurality of payers associated with each basis of comparison.

Information used to determine metrics and/or a percentile rank comparison may include information such as procedure information, discharge information, time to submit claim, time to remit, claim approval/denial rate, cost associated with claim denial, or any other metric capable of being used to determine one or more metrics and/or percentile rank. The procedure information may include one or more of inpatient data and/or outpatient data. At least one of a column, an annual claims number, an annual remit number, and/or an average claim value may be associated with one or more of the inpatient and/or outpatient procedure information.

A payer mix information set may include at least one of inpatient and/or outpatient data relating to one or more of government and/or commercial payers. For example, a payer mix table or dataset may include inpatient and outpatient values for one or more commercial payers such as Aetna, Blue Cross Blue Shield (BCBS), Humana, etc. or combination thereof, and may further include one or more government payers such as a Medicare, TriCare, Medicaid, or combination thereof.

A specialty information set may include one or more medical specialty categories, optionally broken down according to inpatient and/or or outpatient values. One or more sets of information may be visually and/or graphically presented to a user. A variety of information may be tracked and presentable to a user, including per-encounter information, grouped encounter information, practice information, hospital-level information, hospital system-level information, etc. One or more breakdowns of procedures, discharge information, claim submission and denial rates, as well as an estimated cost of denials may be presented.

Business Line Comparison—A measure of the major business lines (specialty) or department within a healthcare entity may be provided using at least a portion of the following metrics and/or a percentile rank comparison.

For example, a business line comparison may include one or more peer group comparisons, a national comparison, and/or a general comparison. Specific comparison values may include, for example, at least one of a discharge value, a claim submission value, a remit receipt value, a denials value, and/or a cash value, although any other value or information capable of comparison may be used without departing from the spirit and scope of the present disclosure. Each comparison value may be compared to a specific hospital, a national average, and/or one or more member of a peer group or a peer group average.

Denials Analysis Comparison—A measure of the major causes of claim denials within a healthcare entity may be provided, for example using at least one of the following metrics and/or a percentile rank comparison. Each exemplary metric is optionally defined according to a plurality of metrics. For example, a healthcare entity's ratings or real-world numbers may be presented for one or more payer entities based on one or more of an authorization rating, a billing rating, a charge capture rating, a coding rating, an eligibility rating, a medical necessity rating, or the like, either alone or in combination with one another. The healthcare entity may be compared against a plurality of other entities, for example one or more payers, where a rating associated with one or more metrics may be broken down according across each payer individually, along with an optional general rating element across the payer industry.

Denials associated with at least one of authorization, billing, charge capture, coding, eligibility, and/or medical necessity may be compared against hospital, national, and peer group information.

### Vitality Score—Credit Score for Hospitals

Healthcare and financial industries need a solution that can measure and compare healthcare provider performance more effectively. Today, many hospitals self-report data which is heavily interpreted and does not tell the whole story.

A Vitality Score may be similar to a consumer's credit score in various aspects. A consumer credit score may be determined according to factors such as whether the consumer pays his or her bills on time, whether a line of credit for the consumer is maxed out, the overall length of credit for the consumer, whether there is any new credit for the consumer, the number of credit accounts for the consumer, the type of accounts held by the consumer, and other factors. A Vitality Score may be based, in whole or in part, upon one or more of payer performance history, claim volume, accounts receivable days, age of cash, a number of claims or remits or ratio thereof, a payer mix value, and/or other factors associated with a healthcare entity, such as a provider, payer, or other entity.

Implementations consistent with the present disclosure may optionally combine two years of healthcare claims 837 and payer remittance 835 data along with bank transaction files to create the ATEX Vitality Score™, although historical information relating to any period of time may be used without departing from the spirit and scope of the present disclosure.

The score may be a composite of four individual indices comprising multiple individual metrics. The individual indices may include one or more of Velocity, Value, Variety, and/or Volatility.

### Velocity Index

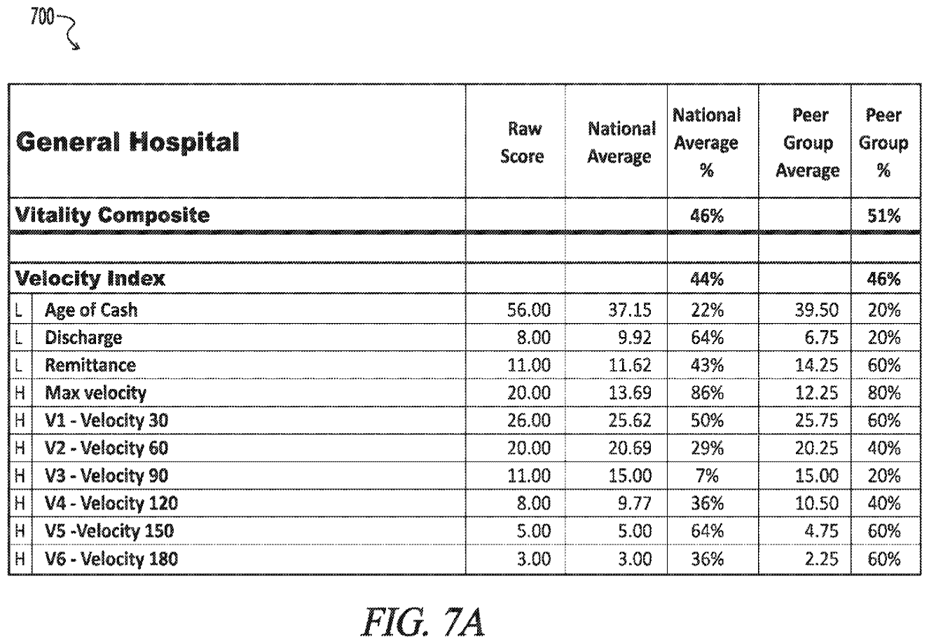

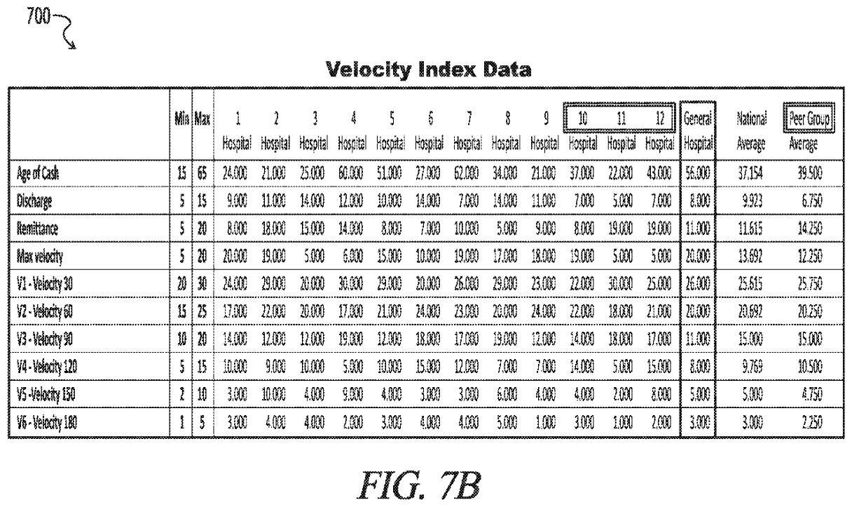

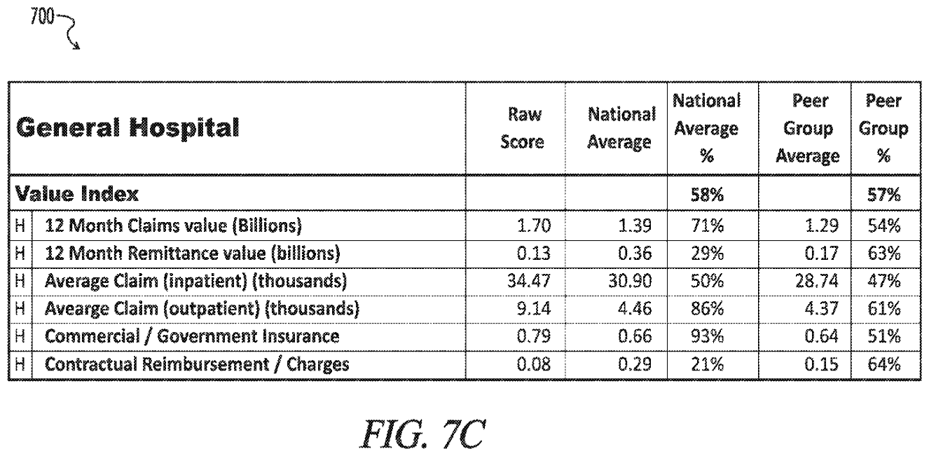

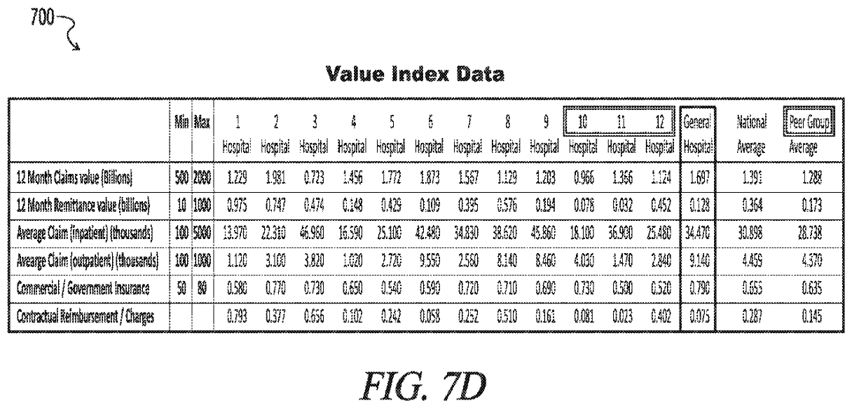

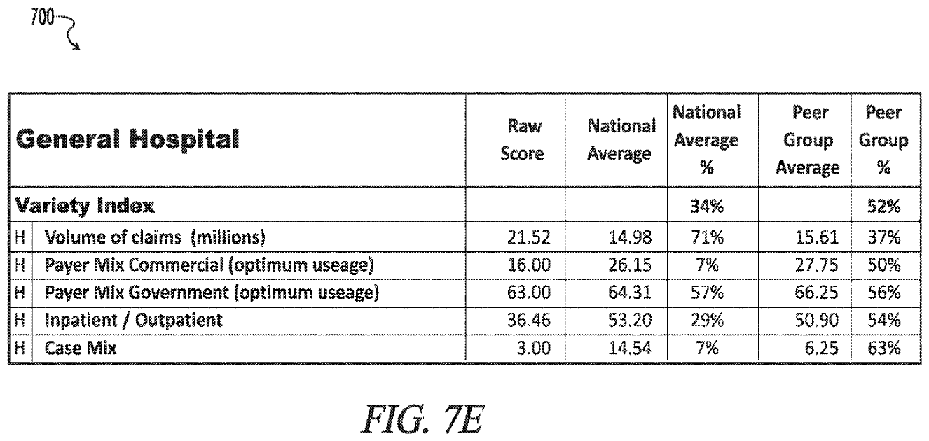

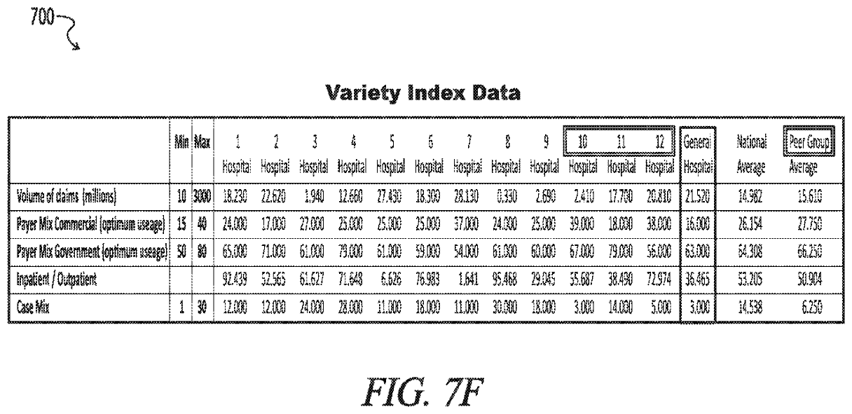

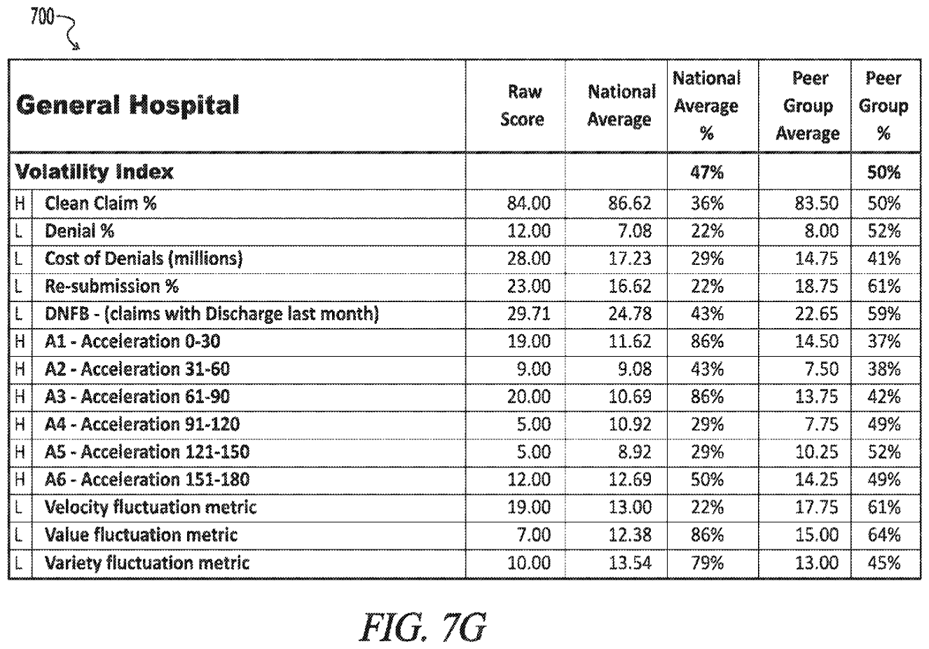

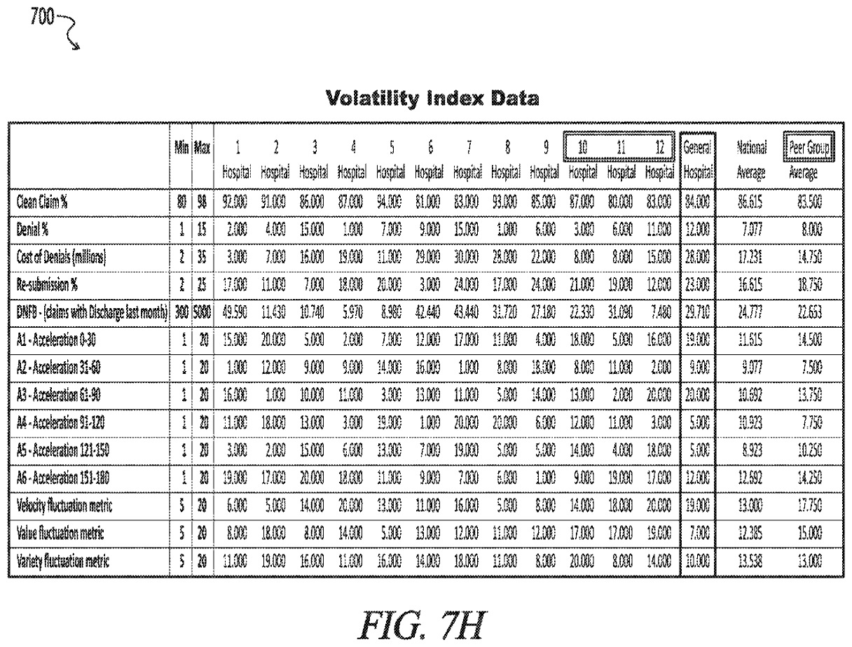

The Velocity Index may be used to measure the speed at which money is collected. Exemplary aspects of the velocity index consistent with the present disclosure are illustrated, for example, by FIG. 7A, with underlying exemplary data illustrated by FIG. 7B.

Values associated with the Velocity Index may include one or more of the following values—

Age of Cash (e.g., measured in days)—may include a weighted average age of cash based at least in part upon the weighted average of each payment for each encounter, then average of all encounters for the designated time period. Age may be calculated at least in part from the beginning date of service until remittance payment date.

Discharge Not Final Billed (e.g., measured in days)-optionally an average count of days between discharge date, and date of claim submission for a designated time period.

Remittance—First payment (e.g., measured in days)—optionally an average count of days from claim submission date until first positive remittance date and for all (or a portion of) submitted claims for the designated time period.

Remittance—Full payment (e.g., measured in days)-optionally an average count of days between claim submission date receipt of payer portion and 90% of patient responsibility amount for the designated time period.

Maximum Velocity of cash collections-optionally an average day of greatest velocity for remittances, beginning with the date of service until first payer payment remittance date based on normalized timeframes for example set to zero days as date of service date.

### Value Index

The Value Index may optionally measure the financial value of reimbursement to a healthcare provider. Exemplary aspects of the value index consistent with the present disclosure are illustrated, for example, by FIG. 7C, with underlying exemplary data illustrated by FIG. 7D.

Values associated with the Value Index may include one or more of the following values—

Average Claim Value—optionally an average amount of all submitted claims by payer and business line.

Ratio of Base Pay—may provide a calculation of reimbursement payment submitted with Medicare reimbursement optionally set as base 1 ratio then reported by payer and business line.

Commercial/Government Insurance (%)—may include an average percentage of total amount of payments with Commercial or Governmental insurance payer.

Contractual Reimbursement/Charges (%)—may be an average percentage of total values of full charges to contractual reimbursement amount.

### Variety Index

The Variety Index may measure the diversification of cashflows by payers and payer types, similar to a diversified financial investment portfolio. Exemplary aspects of the variety index consistent with the present disclosure are illustrated, for example, by FIG. 7E, with underlying exemplary data illustrated by FIG. 7F.

Values associated with the Variety Index may include one or more of the following values—

Payer mix Commercial—may be a percentage of claims by commercial payer class based on % of reimbursement.

Payer mix Government—may include a percentage of claims by payer class government based on % of reimbursement.

Inpatient/Outpatient (%)—may be percentages of claims submitted that are listed as inpatient vs percentage of claims listed as Outpatient.

Case Mix (severity of cases)—optionally the average of the relative value assigned to an inpatient diagnosis-related group based on total activity see CMS data.

Denial (%)—may be an average percentage of claims that have full or partial claim denials.

Cost of Denials (%)—optionally a percentage of claims based on total charge master amount for claims with full or partial denials.

Re-Submission Rate (%)—may be a percentage count of claims that needed more than one claim message for a single claim encounter.

Discharge (Not Final Billed)—Average monthly remittable payment for claim cases that have been discharged but have not yet submitted a claim.

### Volatility Index

The Volatility Index may measure the risk associated with healthcare cash flow, specifically one or more factors affecting payment (such as, e.g., denials). Exemplary aspects of the volatility index consistent with the present disclosure are illustrated, for example, by FIG. 7G, with underlying exemplary data illustrated by FIG. 7H.

Values associated with the Volatility Index may include one or more of the following values—

Denial (%)—may be an average percentage of claims that have full or partial claim denials.

Cost of Denials (%)—may be a percentage of claims based on total charge master amount for claims with full or partial denials.

Re-Submission Rate (%)—may be a percentage count of claims that needed more than one claim message for a single claim encounter.

Discharge Not Final Billed—may include an average monthly remittable payment for claim cases that have been discharged but have not yet submitted a claim.

FIG. 1 illustrates an exemplary embodiment of computer system-level implementation consistent with the present disclosure. The exemplary embodiment illustrated by FIG. 1 includes an end user electronic device 100. The end user electronic device 100 includes one or more of a microprocessor 102, a storage unit 104, a communications unit 106, and/or a display unit 108. The communications unit 106 of the end user electronic device 100 is configured to communicate with a network 110 via a communications link 101. In one exemplary embodiment, the network 110 includes the Internet, a public network, a private network, or any other communications network capable of conveying electronic communications. Connection between the communications unit 106 and network 110 is configured to be performed by wired interface, wireless interface, or a combination thereof, without departing from the spirit and the scope of the present disclosure. In one exemplary operation, the end user electronic device 100 stores one or more sets of instructions in the storage unit 104, which are configured to be executed by the microprocessor 102 to perform operations corresponding to the one or more sets of instructions. The display unit 108 is embodied within the end user electronic device 100 in one embodiment and is configured to be either wired or wirelessly interfaced with the end user electronic device 100.

In various exemplary embodiments, the end user electronic device 100 is at least one of a desktop computer, a laptop computer, a smart phone, or any other electronic device capable of executing instructions. The microprocessor 102 is configured to take the form of a generic hardware processor, a special-purpose hardware processor, or a combination thereof. In embodiments having a generic hardware processor (e.g., as a central processing unit (CPU) available from manufacturers such as Intel and AMD), the generic hardware processor is configured to be converted to a special-purpose processor by means of being programmed to execute and/or by executing a particular algorithm in the manner discussed herein for providing one or more specific operations or results.

The end user electronic device 100 is configured in various embodiments to be associated with a fixed location, but is also capable of being transported, either during operation or while powered off. In one embodiment where the end user electronic device 100 is a client's cellular telephone, the end user electronic device 100 is at least temporarily located at a client's premises. In various embodiments, the end user electronic device 100 is configured to operate remotely and is configured to obtain or otherwise operate upon one or more instructions stored physically remote from the end user electronic device 100 (e.g., via client-server communications and/or cloud-based computing).

The exemplary embodiment illustrated by FIG. 1 includes at least one server 120. Each server 120 is configured to connect to the network 110 via the communications link 121 and includes at least one of a microprocessor 122, a storage unit 124, a communications unit 126, and a display unit 128. Each of the microprocessor 122, the storage unit 124, the communications unit 126, and the display unit 128 is configured to respectively correspond to the previously described microprocessor 102, the storage unit 104, the communications unit 106, and the display unit 108 without departing from the spirit and the scope of the present disclosure.

Each server 120 is configured in various embodiments to be at a fixed physical location and/or may be capable of being transported, either during operation or while powered off. In one embodiment, one or more server 120 is configured to be located at a fixed location and comprises desktop computer or server computer configured to receive input from an end user regarding information discussed above. At least one server 120 may be configured to operate as a standalone server, as a distributed server, as a cloud service-based server, or any other configuration capable of executing or otherwise implementing at least one action. One or more server 120 may have stored therein or otherwise have access to an application or information storage system including information or data executable thereon or therewith to perform one or more operations described herein (including determining one or more values, parameters, data sets, data operations, or any other operation, process, or step consistent with the present disclosure). In various embodiments, the server 120 is configured to operate remotely, and is additionally configured to obtain or otherwise operate upon one or more instructions stored physically remote from the server 120 (e.g., via client-server communications and/or cloud-based computing).

One or more data source 130 consistent with the present disclosure may be provided by one or more electronic device(s) in various exemplary embodiments. Each data source 130 is configured to connect to the network 110 via the communications link 131 and includes at least one of a microprocessor 132, a storage unit 134, a communications unit 136, and a display unit 138. Each of the microprocessor 132, the storage unit 134, the communications unit 136, and the display unit 138 are configured to correspond to the previously described microprocessor 102, the storage unit 104, the communications unit 106, and the display unit 108 without departing from the spirit and the scope of the present disclosure.

Each data source 130 is configured in various embodiments to be associated with a fixed location, but is also capable of being transported, either during operation or while powered off. In one embodiment, one or more data source 130 are configured to be located at a fixed location and to comprise a desktop or server computer. At least one data source 130 may be configured to operate as a standalone server, as a distributed server, as a cloud service-based server, or any other configuration capable of executing or otherwise implementing at least one action. In one exemplary embodiment, at least one of the one or more data source 130 comprises a moveable laptop or tablet computer. In various embodiments, each data source 130 is configured to operate remotely, and is also configured to obtain or otherwise operate upon one or more instructions stored physically remote from the data source 130 (e.g., via client-server communications and/or cloud-based computing). Each data source 130 may be configured to store at least one set of information (e.g., via the storage unit 134) and to selectively convey at least a portion of the at least one set of information, for example via the network 110.

In one exemplary embodiment, one or more third-party system 140 consistent with the present disclosure are provided by one or more electronic devices. In one exemplary embodiment, one or more third-party system 140 may be associated with one or more providers of information, such as insurers, payers, hospitals, service providers, or any other source of third-party information (and combination(s) thereof). Each third-party system 140 may be configured to connect to the network 110 via the communications link 141 and is configured to include at least one of a microprocessor 142, a storage unit 144, a communications unit 146, and a display unit 148. Each of the microprocessor 142, the storage unit 144, the communications unit 146, and the display unit 148, in one exemplary embodiment, corresponds to the previously described microprocessor 102, storage unit 104, communications unit 106, and/or display unit 108, without departing from the spirit and the scope of the present disclosure.

One or more third-party system 140 may be configured to operate as a standalone server, as a distributed server, as a cloud service-based server, or any other configuration capable of executing or otherwise implementing at least one action associated with a third-party system. When implemented in a distributed manner, one or more of the third-party system 140 may be connected to the network 110 and/or to a separate (e.g., private) network. In various embodiments, at least one third-party system 140 is configured to operate remotely and is further configured to obtain or otherwise operate upon one or more instructions stored physically remote from the third-party system 140 (e.g., via client-server communications and/or cloud-based computing).

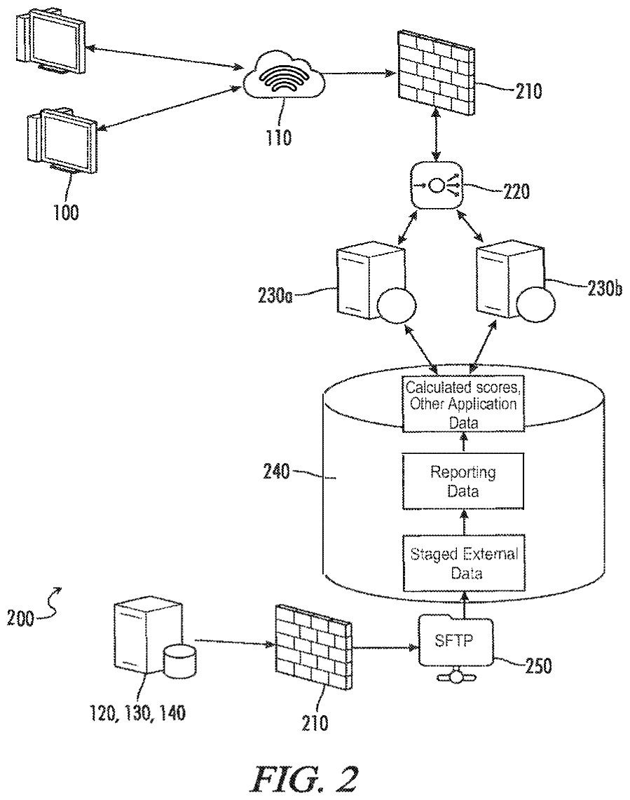

FIG. 2 illustrates a functional network diagram of an exemplary embodiment of a system according to aspects of the present disclosure. One or more end users may interact with one or more end user electronic devices 100 (e.g., end user electronic devices 100), for example at a hospital or doctor's location (although one or more end users may be capable of interacting with an end user electronic device 100 outside of the physical confines of a hospital or doctor's office, such as via a mobile electronic device of electronic device located at the user's residence). The end user electronic devices 100 may be capable of communicating through the network 110 (e.g., public Internet and optionally through one or more of a network firewall 210 and/or load balancer 220 with one or more web servers 230a, 230b (e.g., at least one server 120, data source 130, and/or third-party system 140)). The one or more web servers may include or otherwise be coupled to a data storage (e.g., a data source 130). The data storage and/or other data storage associated with a server 120 and/or third-party system 140 may include information 240 such as calculated scores and/or other application data, reporting data, and/or staged external data (although other data may be stored by or otherwise accessible to the data storage). The data storage may be configured to receive information stored by the data storage from at least one external claims and remit data system (e.g., operating as a third-party system 140). Although the term third-party is used it should be appreciated that one or more third-party system 140 which may be a provider system's device or a data source associated with a healthcare provider which stores provider information may include or otherwise have access to non-third-party data systems or information; Furthermore, despite being referred to as third-party system, at least one system 140 may be part of overall system described herein and/or may be part of or otherwise associated with a data source 130. The external claims and remit data may be communicated to the data storage optionally through at least one of a network firewall 210 and/or Secure File Transfer Protocol (SFTP) interface 250. Although described with reference to the SFTP protocol, it should be appreciated that any secure communication protocol may be implemented without departing from the spirit and scope of the present disclosure.

In an exemplary implementation, the Vitality Score may be accessed via the internet (e.g., via the network 110) as a Software As A Service (SAAS) solution. Users may access a secure, encrypted, website URL (SHTTP) through a desktop PC, laptop PC, tablet or mobile device with a given username and password. The user may be assigned to a user type which may define what kind of data, facility, and analytics they can access. A typical user might be a hospital or bank employee who may have access to the entire healthcare system, a regional group, or specific, individual hospitals. Upon logging into the SHTTP website via the server 120, the data storage 130, and/or third-party system 140, the user's credentials define access to hospital(s) are presented on the screen (e.g., via the display unit 108 thereof). User access may be administratively established in the database where the healthcare system, regional groups of hospitals, and individual hospitals are defined. Demographic information may be loaded into the solution about the hospital (e.g., address, hospital type, financial data, healthcare service data, etc.).

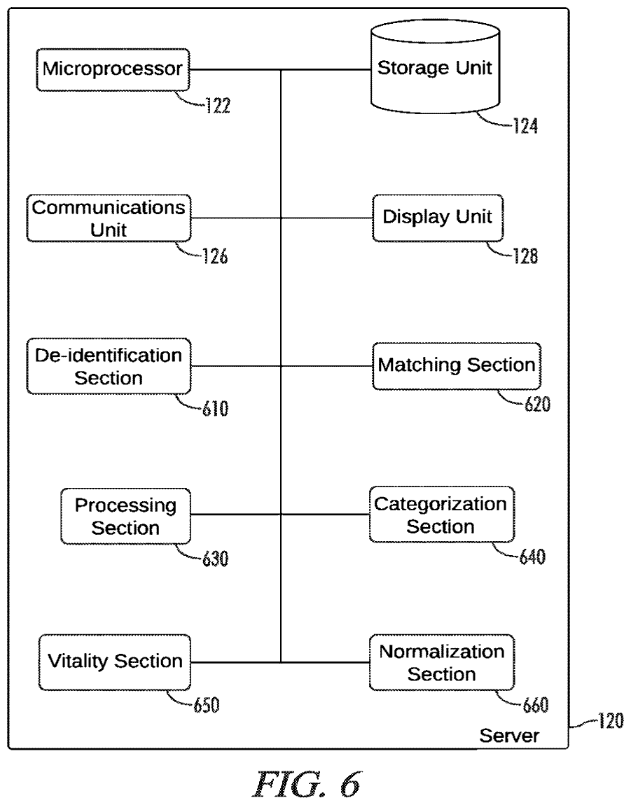

Hospitals may provide the system with two years of historical claims (EDI 835) and remittance (EDI 837) data files in an exemplary embodiment (although any length of historical data may be used without departing from the spirit and scope of the present disclosure). This historical claims and payment information may be loaded into the Vitality Score and may be pre-processed for analytics (e.g., as described herein with reference to one or more operations of the server 120, for example using the Vitality Section 650). The Vitality Score may be used to calculate a plurality (e.g., over 25) pre-defined metrics measuring the velocity, value, variety, and volatility of cash. These metrics may help define how efficient a hospital submits claims to an insurance company, how fast the insurance company pays the hospital, how often a claim is denied, and ultimately how much the hospital is paid. It may calculate the portfolio of payments from different insurance companies and can even analyze payments and denials down to a clinical specialty level (cardiology, orthopedics, OB, etc.). The combination of all these individual metrics are optionally summarized as indices for Velocity, Value, Variety, and Volatility. The indices may then be consolidated into an overall Vitality Score in various embodiments, for example by the Vitality Section 650 described herein with reference to FIG. 6.

When a user signs into the SHTTP website (e.g., via the server 120), they can select which hospital or groups of hospitals they want to analyze. Users can select from a variety of pre-defined analytics of the ATEX Vitality Score™. Users can compare a single hospital from a large health system to its peers within the same health system using one or more sets of information and/or visual elements provided to the user via the display unit 108 of the user's device 100 as received from the server 120 via the network 110. Users can also compare hospitals to national and peer groups outside their health system. This may be accomplished by deidentifying and aggregating individual hospital claims and remittance information into a centralized repository (e.g., as stored or accessible to the server 120, either in whole or in part). The ATEX Vitality Score™ can assimilate the Nation's 3,500+ hospitals into one central repository thereby empowering hospital, banks, consultants, insurance companies, etc. with transparent information to improve the healthcare industry.

Users can select their hospitals and compare their performance against other hospitals based on the demographics loaded into the database. Matrixed with the various pre-defined and pre-calculated metrics, users can select which analyses to perform and compare. Users can select to compare reimbursement rates by hospital by insurance company, reimbursement by clinical specialty by insurance company, denials by hospital by insurance company, etc. This level of analysis can be accomplished because all the hospitals' claims and remittance data is loaded, aggregated, and processed making it searchable for comparison.

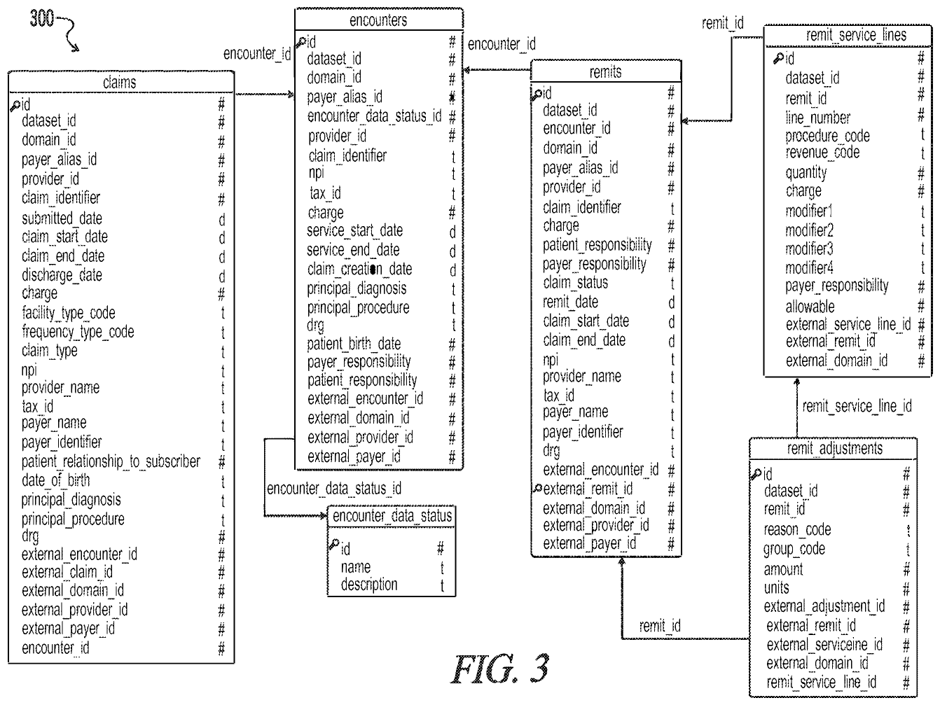

FIG. 3 illustrates an exemplary embodiment of report data tables 300 according to aspects of the present disclosure. In various embodiments, one or more report data tables 300 may be used to store a normalized representation of a patient encounter. This patient encounter information may include data sets relating to one or more of claims data, encounter data, encounter status data, remit data, remit service fine data, and/or remit adjustment data (although additional or fewer data sets may be used). Each of the one or more of claims data, encounter data, encounter status data, remit data, remit service fine data, and/or remit adjustment data sets may include one or more subsets of data, for example as included in the embodiment illustrated by FIG. 3. In various embodiments, at least a portion of the information illustrated by FIG. 3 may be stored by or otherwise accessible to one or more server 120 and/or data source 130.

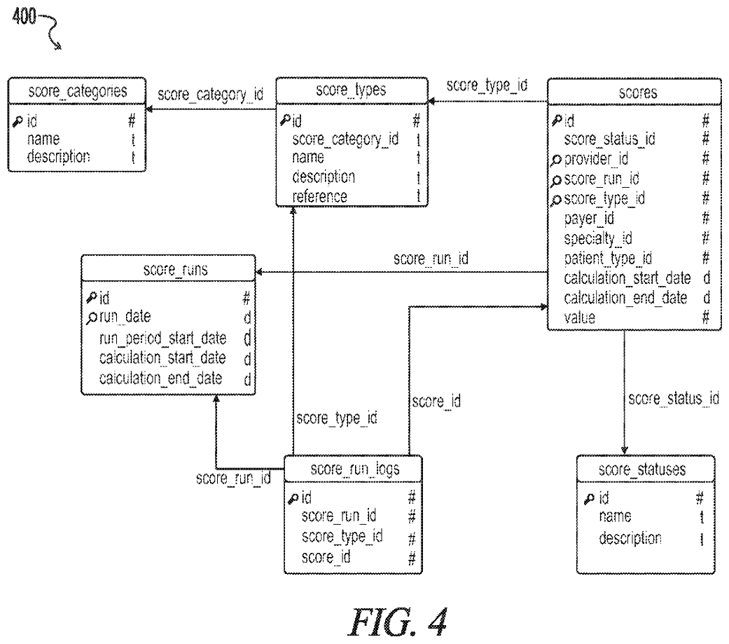

FIG. 4 illustrates an exemplary embodiment of a table structure 400 including pre-calculated scores according to an exemplary embodiment. In various embodiments, the table structure 400 may include one or more sets of data such as score category data, score type data, score data, score run data, score run log data, and/or score status data (although additional or fewer data sets may be used). Each of the one or more of score category data, score type data, score data, score run data, score run log data, and/or score status data sets may include one or more subsets of data, for example as included in the embodiment illustrated by FIG. 4. In various embodiments, at least a portion of the information illustrated by FIG. 4 may be stored by or otherwise accessible to one or more server 120 and/or data source 130.

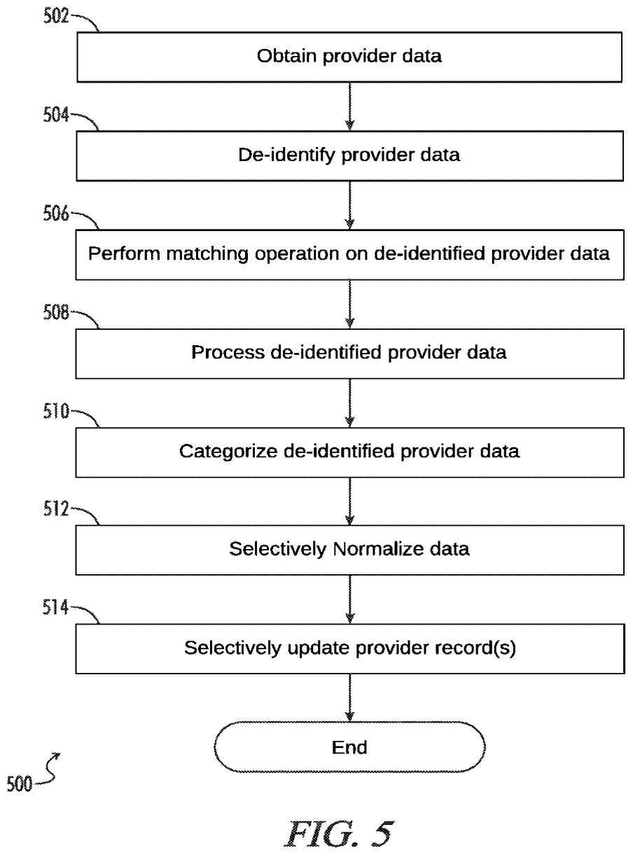

FIG. 5 illustrates an exemplary embodiment of a process for processing provider data according to aspects of the present disclosure. The process 500 begins at an operation 502 where provider data is obtained. The obtained provider data may be obtained directly from a provider or from a storage location associated with a provider, for example via the network 110. Provider data may include any service or financial information relating to the provider, such as 837 claim files, 835 remittance files, financial transaction data, or the like. After obtaining the provider data, the provider data may be de-identified at an operation 504. The provider data may be de-identified based at least in part upon a known data format or content according to an accepted medical information format. A matching operation may be performed on at least a portion of the de-identified provider data at operation 506 to match provider data to an encounter or to define a new encounter for the provider. As used herein, an encounter may correspond to a specific interaction or service performed by or in conjunction with the provider, such as a patient treatment or service. One or more encounters may be matched using one or more second data sets, such as remittance data associated with an 835 remittance file.

The process then continues to an operation 508 where de-identified provider data is processed. Processing at operation 508 may include decoding the de-identified provider data according to a predetermined format or coding, or additionally or alternatively may include dynamically based processing using a self-learning algorithm, artificial intelligence, and/or static or dynamic mapping data associated with at least a portion of the de-identified provider data being processed.

The process then continues to an operation 510 where de-identified provider data is categorized. Categorization at operation 510 may include associating at least a portion of the de-identified provider data, such as a predefined or known code, with one or more categories or associations. Categorization at operation 510 may be a one-to-one, a one-to-many-, or a many-to-one mapping. For example, a single set of coded information derived from the de-identified provider data may be associated with a plurality of categories in a predefined and/or dynamically determined manner.

After categorization at operation 510 the process continues to an operation 512 where one or more sets of de-identified provider data and/or categorized de-identified provider data is selectively normalized. Normalization may include, for example, cost of living adjustments, regional pricing modification, procedure-based modification, or other form of normalizing at least one set of information across a national, regional, or peer group. The process 500 continues to an operation 514 where one or more provider records may be selectively updated, for example in situations where provider information, payer information, and/or payment information is updated, newly presented, or removed (for example, in the context of an encounter). The process may then end.

FIG. 6 illustrates an exemplary embodiment of a partial block diagram of a server according to aspects of the present disclosure. The server 120 may include each element previously described herein with reference to FIG. 1. The server 120 may further include one or more of a de-identification section 610, a matching section 620, a processing section 630, a categorization section 640, a vitality section 650, and/or a normalization section 660. Although illustrated as separate sections, it should be appreciated that one or more of the identified sections may be performed by a single or plurality of elements without departing from the spirit and scope of the present disclosure. The de-identification section 610 may be configured to perform a de-identification operation on at least a portion of a received set of provider data (e.g., received from a healthcare provider via the network 110). The matching section 620 may be configured to perform a matching operation on the at least a portion of the received set of provider data having been de-identified by the de-identification section to create a set of matched de-identified data. The processing section 630 may be configured to process the matched de-identified data to extract at least one set of coded information according to a predetermined data format of the set of provider data. The categorization section 640 may be configured to process the matched de-identified data to extract at least one set of coded information according to a predetermined data format of the set of provider data. The vitality section 650 may be a vitality scoring section which is configured to determine at least one vitality composite score based at least in part upon the categorized at least one set of coded information. The normalization section 660 may be configured to perform a normalization operation on the at least one set of coded information in association with the vitality score to form a set of normalized provider data. The server 120 may include a portal section (not illustrated) which is capable of providing an interface for interacting with a user, for example via the network 110.

FIGS. 7A-7H illustrate an exemplary embodiment of a Vitality Score table 700 including Volatility Index, Value Index, Variety Index, and Volatility Index data for a plurality of hospitals, peer groups, and national data, along with corresponding raw data, according to aspects of the present disclosure. FIGS. 7A and 7B represent matched categorized data and corresponding raw data for the velocity index, FIGS. 7C and 7D represent matched categorized data and corresponding raw data for the value index, FIGS. 7E and 7F represent matched categorized data and corresponding raw data for the variety index, and FIGS. 7G and 7H represent matched categorized data and corresponding raw data for the volatility index. The raw data includes data for a plurality of hospitals, along with peer group average data and national average data.

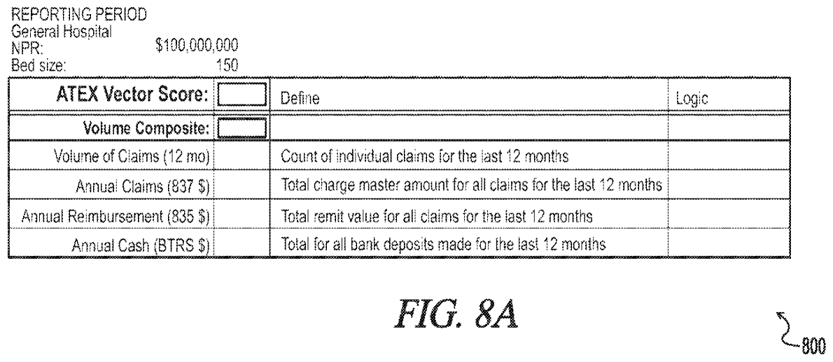

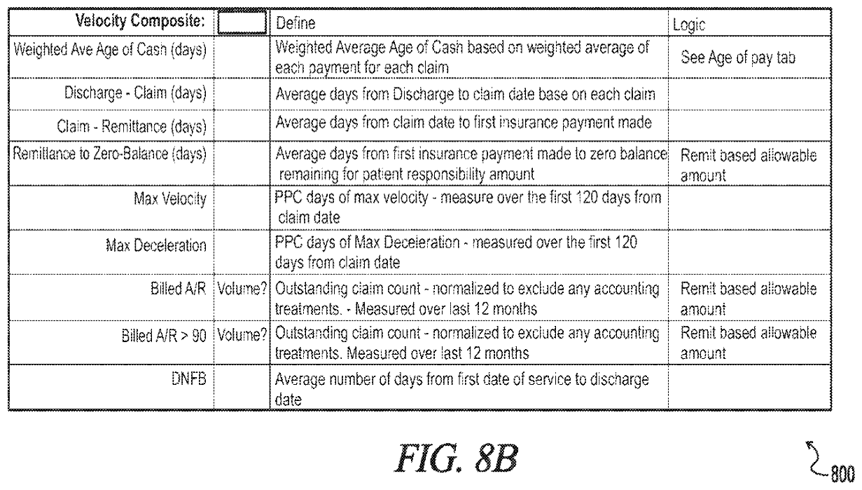

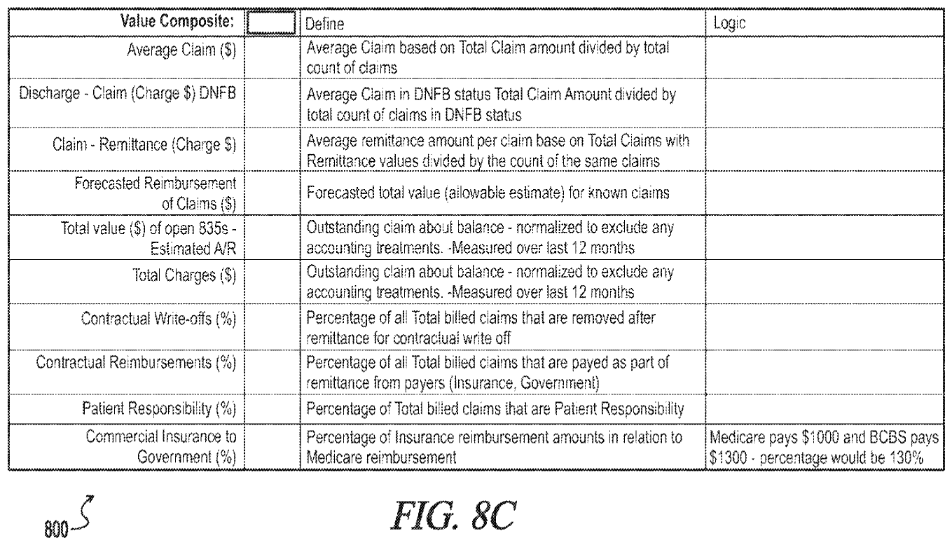

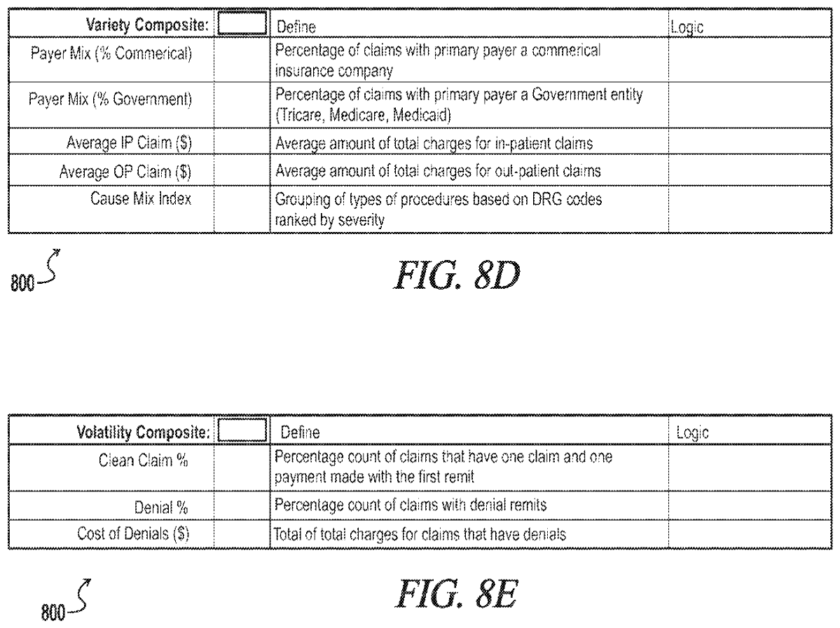

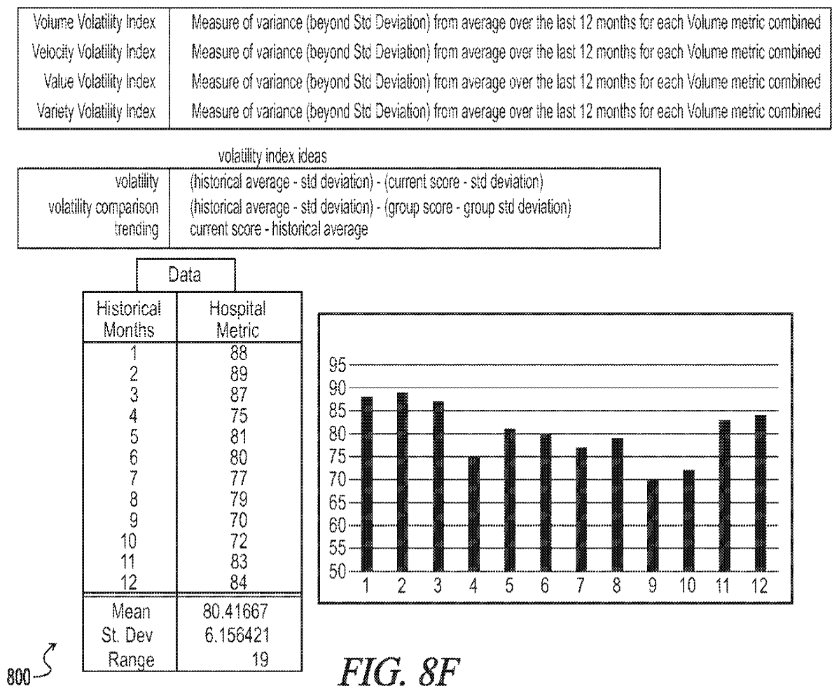

FIGS. 8A-8F illustrate an exemplary embodiment of a vector scoring metric scenario 800 including an ATEX Vector Score, a volume composite, a velocity composite, a value composite, a variety composite, and a volatility composite, along with sub-components thereof for each composite value according to aspects of the present disclosure and consistent with the present disclosure.

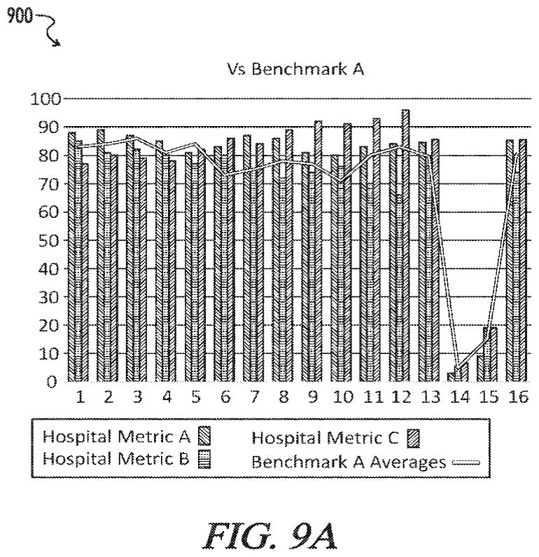

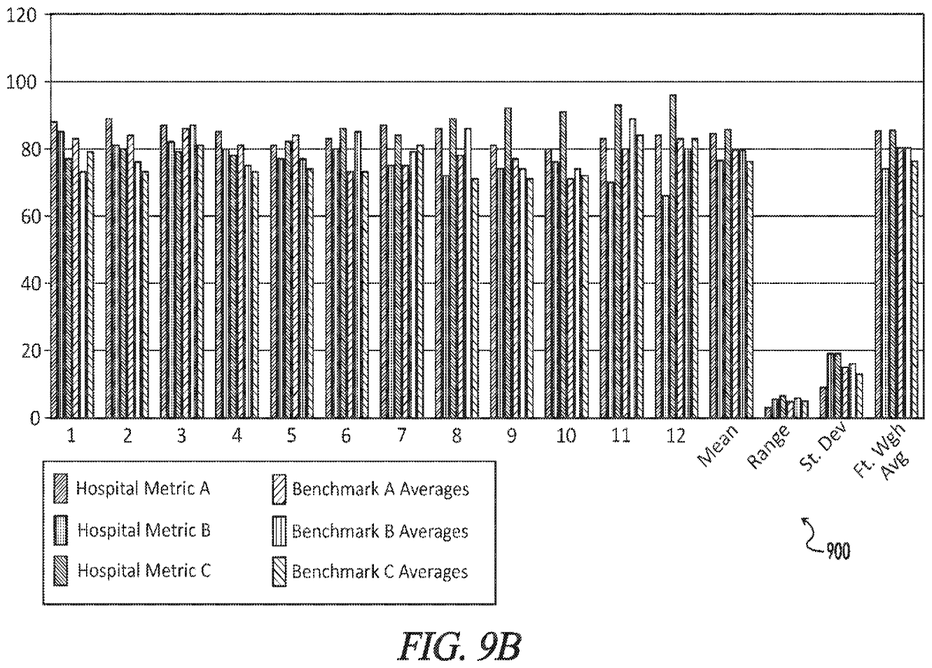

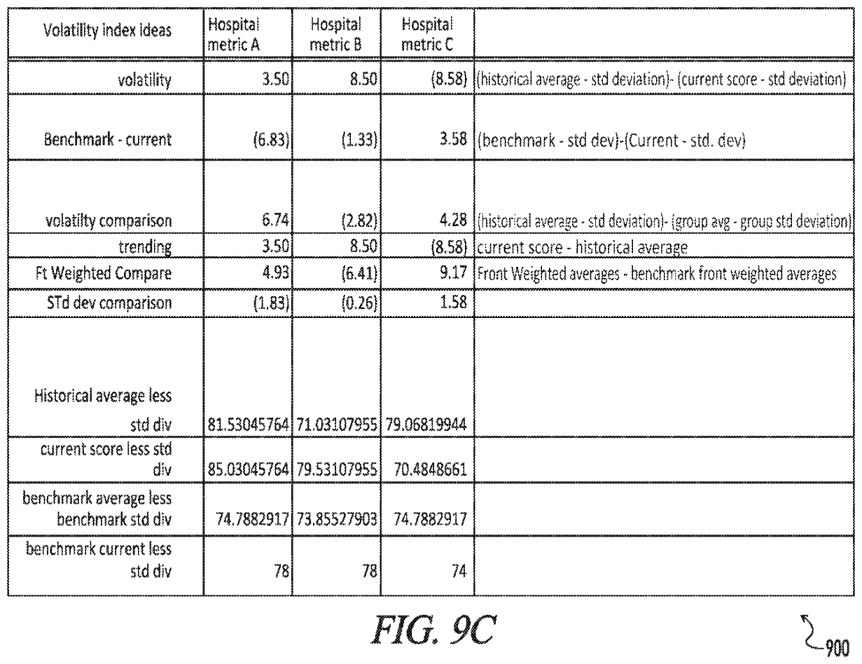

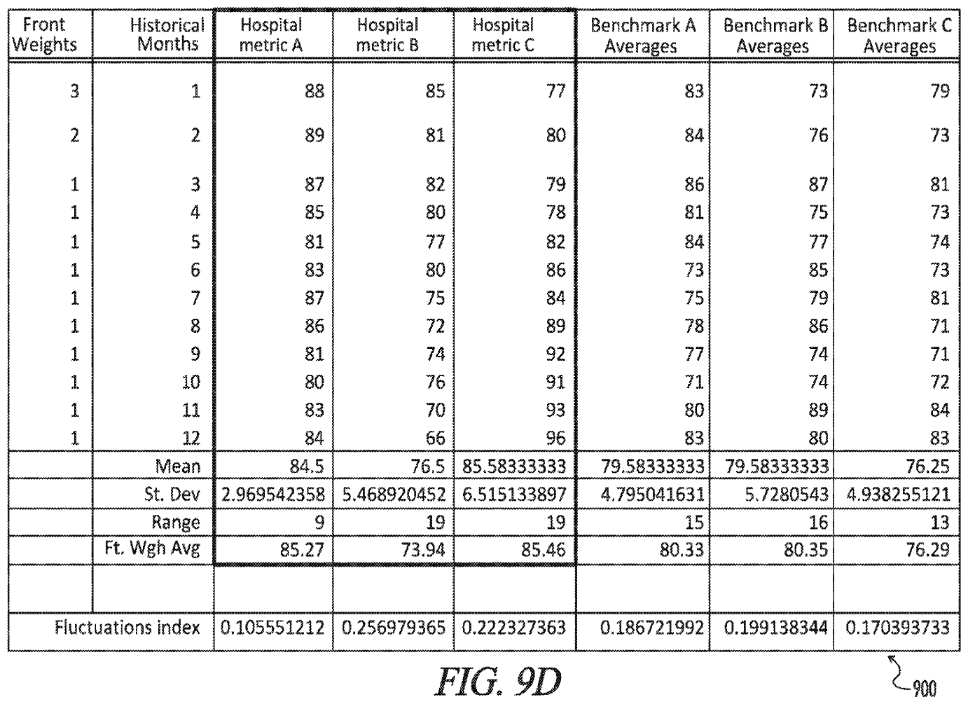

FIGS. 9A-9D illustrate an exemplary embodiment of Volatility Index calculation and presentation capabilities 900 to a user according to aspects of the present disclosure. FIGS. 9A-9D illustrate hospital and benchmark comparison data and visuals presentable to a user for use with the present disclosure.

To facilitate the understanding of the embodiments described herein, a number of terms are defined below. The terms defined herein have meanings as commonly understood by a person of ordinary skill in the areas relevant to the present invention. Terms such as “a,” “an,” and “the” are not intended to refer to only a singular entity, but rather include the general class of which a specific example may be used for illustration. The terminology herein is used to describe specific embodiments of the invention, but their usage does not delimit the invention, except as set forth in the claims. The phrase “in one embodiment,” as used herein does not necessarily refer to the same embodiment, although it may.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

The previous detailed description has been provided for the purposes of illustration and description. Thus, although there have been described particular embodiments of a new and useful invention, it is not intended that such references be construed as limitations upon the scope of this invention except as set forth in the following claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. A system for determining healthcare vitality, comprising:
a network;
a plurality of healthcare provider devices, each healthcare provider device of the plurality of healthcare provider devices including a communication section and being communicatively coupled to the network via the communication section, wherein each healthcare provider device of the plurality of healthcare provider devices is associated with a healthcare provider and configured to transmit provider data via the network, the provider data comprising information regarding a medical service performed by the healthcare provider;
a server having a storage having a communication unit, the server communicatively coupled to the network via the communication unit and configured to:
receive provider data from the plurality of healthcare provider devices;
de-identify at least a portion of the provider data received from the plurality of healthcare provider devices based at least in part on a known data format;
match at least a portion of the provider data having been de-identified to encounter data to create a set of matched de-identified data, the encounter data being defined by one or more encounters, each of the one or more encounters corresponding to other medical services performed by the healthcare provider or a third-party provider;
extract, from the matched de-identified data, at least one set of coded information according to a predetermined data format of the set of provider data to categorize the matched de-identified data based at least in part upon the determined at least one set of coded information;
determine a vitality composite score based at least in part upon the at least one set of coded information, the vitality composite score comprising at least one of a velocity index, a value index, a variety index, or a volatility index;
selectively normalize at least one set of coded information in association with the vitality composite score to form normalized provider data, the normalized provider data representative of provider data grouped on a national, regional, or peer basis; and
compare normalized provider data for a selected healthcare provider to normalized provider data for one or more healthcare providers to create comparison data.

20. The system of claim 19, wherein the server is further configured to associate one or more of the categorized matched de-identified data with the plurality of healthcare providers.

21. The system of claim 19, further comprising:
a user device including a communication section and a display unit, the user device communicatively coupled to the network via the communication section and configured to provide a representation of the information relating to the normalized provider data via the display unit.

22. The system of claim 21, wherein the user device is configured to provide a user with access to information relating to normalized provider data for the selected healthcare provider.

23. The system of claim 21, wherein the user device is further configured to display the normalized provider data for a plurality of healthcare providers via the display unit.

24. The system of claim 21, wherein the user device is configured to provide a user with access to information relating to the normalized provider data for the selected healthcare provider via a secure, encrypted, website URL (SHTTP), as made available by the server via the network.

25. The system of claim 21, wherein the server is further configured to assign each user of the user device a user type, the user type being associated with the user's level of access to data stored on the server.

26. The system of claim 19, wherein the server is further configured to categorize the at least a portion of the matched de-identified data to at least one category which is non-coded with respect to a data format of the provider data.

27. The system of claim 19, wherein the provider data includes 837 claim files or 835 remittance files.

28. The system of claim 19, wherein the encounter data includes at least one or more of claims data, encounter status data, remit data, remit service fine data, or remit adjustment data.

29. The system of claim 19, wherein the velocity index comprises a measurement of how quickly the healthcare provider is expected to collect reimbursement for performance of medical services, the value index comprises a measurement of a financial value of reimbursement to the healthcare provider, the variety index comprises a measurement of a diversification of cashflow from a plurality of payers, and the volatility index comprises a measurement risk associated with denials flowing to the healthcare provider.

30. The system of claim 19, wherein the normalized provider data for the one or more healthcare providers is stored in the storage of the server.

31. A method for determining healthcare vitality, comprising:
receiving a plurality of a set of provider data on a server, each set of provider data being received from a healthcare provider device and comprising information regarding a medical service performed by the healthcare provider, the healthcare provider device and the server being communicatively coupled via a network;
de-identifying at least a portion of each received set of provider data based at least in part on a known data format;
matching at least a portion of each set of provider data having been de-identified to encounter data to create a set of matched de-identified data, the encounter data being defined by one or more encounters, each of the one or more encounters corresponding to other medical services performed by the healthcare provider or a third-party provider;
extracting at least one set of coded information from the matched de-identified data according to a predetermined data format of the set of provider data to categorize the matched de-identified data based at least in part upon the determined at least one set of coded information;
determining a vitality composite score based at least in part upon the at least one set of coded information, the vitality composite score comprising at least one of a velocity index, a value index, a variety index, or a volatility index;
selectively normalizing at least one set of coded information in association with the vitality composite score to form normalized provider data, the normalized provider data representative of provider data grouped on a national, regional, or peer basis; and
comparing normalized provider data for a selected healthcare provider to normalized provider data for one or more healthcare providers to create comparison data.

32. The method of claim 31, further comprising:
associating one or more categorized matched de-identified data with the plurality of healthcare providers, wherein the one or more categorized matched de-identified data relate to the vitality composite score for the plurality of healthcare providers or payers.

33. The method of claim 31, further comprising:
selectively transmitting information relating to normalized provider data for the selected healthcare provider and the comparison data to a user device, the user device being communicatively coupled to the network.

34. The method of claim 33, further comprising:
visually displaying the information relating to the normalized provider data for the selected healthcare provider and the comparison data on a display unit of the user device.

35. The method of claim 33, wherein the user device is configured to provide a user with access to information relating to the normalized provider data for the selected healthcare provider and the comparison data via a secure, encrypted, website URL (SHTTP), as made available by the server via the network.

36. The method of claim 33, further comprising:
assigning each user of the user device a user type, the user type being associated with the user's level of access to data stored on the server.

37. The method of claim 31, wherein each set of provider data includes 837 claim files or 835 remittance files.

38. The method of claim 31, wherein the velocity index comprises a measurement of how quickly the healthcare provider is expected to collect reimbursement for performance of medical services, the value index comprises a measurement of a financial value of reimbursement to the healthcare provider, the variety index comprises a measurement of a diversification of cashflow from a plurality of payers, and the volatility index comprises a measurement risk associated with denials flowing to the healthcare provider.

39. The method of claim 31, wherein the encounter data includes claims data, encounter status data, remit data, remit service fine data, and/or remit adjustment data.
